(19) United States
(12) Patent Application Publication
(10) Pub. No.: US 2026/0168977 A1
(43) Pub. Date: Jun. 18, 2026

(54) SYSTEM AND METHOD FOR EARLY GAS DETECTION IN A PORTABLE GAS DETECTOR WITH A MEASURABLE SENSOR RESPONSE

(71) Applicant: Molex, LLC, Lisle, IL (US)

(72) Inventors: Brian Keith Wrazen, Cheektowaga, NY (US); Shane Lee McEwen, Calgary (CA); Swapnil Sanjay Deore, Williamsville, NY (US); Jonathan Joseph Itschner, Buffalo, NY (US); Benjamin Samuel Madoff, Buffalo, NY (US)

(21) Appl. No.: 19/380,034

(22) Filed: Nov. 5, 2025

Related U.S. Application Data
(60) Provisional application No. 63/733,634, filed on Dec. 13, 2024.

(51) Int. Cl. G01N 33/00

(52) U.S. Cl. CPC G01N 33/0067; G01N 33/0063; G01N 33/007

(57) ABSTRACT

A gas detection system and early warning method utilize normalized data to determine a sample coefficient used to estimate an actual concentration of a gas in the early stages of gas exposure.

### FIELD OF THE INVENTION

This disclosure relates to the field of gas detection. Specifically, this disclosure relates to gas detection with portable gas detector. More specifically, in some embodiments, this disclosure may relate to a method of early gas detection for early warning systems and bump testing of gas detectors.

### BACKGROUND

Many manufacturers have alarms that vary from low level gas exposures to life threatening levels. OSHA and NIOSH have exposure levels recommended to keep employees safe. The seriousness of detecting harmful gas at the earliest time has driven electrochemical sensor manufacturers to create the fastest response sensor possible. This response is indicated by an industry standard T90 value, which is the time the sensor takes to reach 90% of the gas exposure level. The major gas detector manufacturers on the market have advertised T90 response times from 15 to 30 seconds. The T90 response time is considered as a safety factor when purchasing.

Gas detectors have alarm setpoints in ppm (parts per million) of gas concentration for the purpose of warning the user. When the setpoint is reached the device alarms accordingly. The time it takes to get to the warning is proportional to the characteristic T90 response of the electrochemical sensor. The time savings provided an early warning method capable detecting a concentration of gas ahead of the T90 response is particularly valuable as a safety feature in higher concentration events, and as a way to conserve gas, time, and money in the bump testing of gas detectors.

### SUMMARY

The present disclosure includes one or more of the features recited in the appended claims and/or the following features which, alone or in any combination, may comprise patentable subject matter.

According to a first aspect of the present disclosure, a method of determining a concentration of a gas includes detecting a presence of a detected gas with a gas detector and triggering a timer when a detected concentration of the gas reaches a predetermined level. The method includes measuring the concentration of the gas at a first time. The method includes measuring the concentration of the gas at a second time at an end of the timer. The method includes determine a rate of change of the concentration of the gas from the first time to the second time, comparing the rate of change to a known standard, and determining, from the comparison of the rate of change to the known standard, an estimated actual concentration of the detected gas.

According to some embodiments of the first aspect, the first time corresponds to a time when the timer is triggered.

According to some embodiments of the first aspect, the second time is one second after the first time.

According to some embodiments of the first aspect, the predetermined level corresponds to 20% of an actual concentration of the detected gas.

According to some embodiments of the first aspect, the step of triggering the timer when the detected concentration of the gas reaches the predetermined level includes triggering the timer when a measured ADC reaches a predetermined ADC value.

According to some embodiments of the first aspect, the step of comparing the rate of change to the known standard includes obtaining the known standard from a normalized curve for a known gas concentration. The step of obtaining the known standard from the normalized curve may include deriving a standard curve by applying a tangent line approximation to the normalized curve.

According to some embodiments of the first aspect, the step of comparing the rate of change to the known standard includes determining a rate of change of the concentration of the detected gas between the first time and the second time. The step of comparing the rate of change to the known standard may include comparing the rate of change to a standard curve derived from the known standard. The step of determining the estimated actual concentration of the detected gas may include projecting a path of the rate of change up the standard curve of the know standard to obtain the estimated actual concentration.

According to some embodiments of the first aspect, the method includes using the estimated actual concentration to trigger an alarm when the estimated actual concentration is above a predetermined threshold level. The method may include triggering the alarm before the detected gas is measured to be at an actual concentration of the detected gas by a sensor of the gas detector. The method may be used to bump test the gas detector. The estimated actual concentration may be determined within 5 seconds of gas exposure. The alarm may be triggered to signal the estimated actual concentration is above the predetermined threshold level with less than 5 seconds of gas exposure.

According to some embodiments of the first aspect, the step of determining the estimated actual concentration of the detected gas includes taking into account a modifier coefficient identified from the determined rate of change. The step of determining the estimated actual concentration of the detected gas may include taking into account a time variance due to a sensitivity of a sensor of the gas detector.

According to a second aspect of the present disclosure, a method of bump testing a gas detection system includes exposing a sensor of the gas detection system to a calibration gas and detecting a presence of the calibration gas with a sensor of the gas detection system. The method includes triggering a timer when a detected concentration of the calibration gas reaches a predetermined level. The method includes measuring the concentration of the calibration gas at a first time. The method includes measuring the concentration of the calibration gas at a second time at an end of the timer. The method includes determine a rate of change of the concentration of the calibration gas from the first time to the second time, comparing the rate of change to a known standard, and determining, from the comparison of the rate of change to the known standard, an estimated actual concentration of the calibration gas. The method includes triggering a response of the gas detection system when the estimated actual concentration of the calibration gas exceeds a predetermined setpoint.

According to some embodiments of the second aspect, the first time corresponds to a time when the timer is triggered.

According to some embodiments of the second aspect, the second time is one second after the first time.

According to some embodiments of the second aspect, the predetermined level corresponds to 20% of an actual concentration of the calibration gas. The step of triggering the timer when the detected concentration of the calibration gas reaches the predetermined level may include triggering the timer when a measured ADC reaches a predetermined ADC value. The step of comparing the rate of change to the known standard may include obtaining the known standard from a normalized curve for a known gas concentration. The step of obtaining the known standard from the normalized curve may include deriving a standard curve by applying a tangent line approximation to the normalized curve.

According to some embodiments of the second aspect, the step of comparing the rate of change to the known standard includes determining a rate of change of the concentration of the calibration gas between the first time and the second time. The step of comparing the rate of change to the known standard may include comparing the rate of change to a standard curve derived from the known standard. The step of determining the estimated actual concentration of the calibration gas may include projecting a path of the rate of change up the standard curve of the know standard to obtain the estimated actual concentration.

According to some embodiments of the second aspect, the estimated actual concentration is determined within 5 seconds of gas exposure.

According to some embodiments of the second aspect, the alarm is triggered to signal the estimated actual concentration is above the setpoint with less than 5 seconds of gas exposure.

According to some embodiments of the second aspect, the step of determining the estimated actual concentration of the detected gas includes taking into account a modifier coefficient identified from the determined rate of change.

According to some embodiments of the second aspect, the step of determining the estimated actual concentration of the detected gas includes taking into account a time variance due to a sensitivity of a sensor of the gas detector.

According to a third aspect of the present disclosure, a method of determining a concentration of a gas includes detecting a presence of a detected gas with a gas detector and triggering a timer when a detected concentration of the gas reaches a predetermined level. The method includes measuring the concentration of the gas at a first time. The method includes measuring the concentration of the gas at a second time at an end of the timer. The method includes determine a rate of change of the concentration of the gas from the first time to the second time, comparing the rate of change to a known standard, and determining, from the comparison of the rate of change to the known standard, an estimated actual concentration of the detected gas. The method includes trigger an early warning alarm when the estimated actual concentration is above a predetermined threshold level.

According to a third aspect of the present disclosure, the method includes triggering the alarm before the detected gas is measured to be at an actual concentration of the detected gas by a sensor of the gas detector.

According to a third aspect of the present disclosure, the first time corresponds to a time when the timer is triggered.

According to a third aspect of the present disclosure, the second time is one second after the first time.

According to a third aspect of the present disclosure, the predetermined level corresponds to 20% of an actual concentration of the detected gas.

According to a third aspect of the present disclosure, the step of triggering the timer when the detected concentration of the gas reaches the predetermined level includes triggering the timer when a measured ADC reaches a predetermined ADC value.

According to a third aspect of the present disclosure, the step of comparing the rate of change to the known standard includes obtaining the known standard from a normalized curve for a known gas concentration. The step of obtaining the known standard from the normalized curve may include deriving a standard curve by applying a tangent line approximation to the normalized curve.

According to a third aspect of the present disclosure, the step of comparing the rate of change to the known standard includes determining a rate of change of the concentration of the detected gas between the first time and the second time. The step of comparing the rate of change to the known standard may include comparing the rate of change to a standard curve derived from the known standard. The step of determining the estimated actual concentration of the detected gas may include projecting a path of the rate of change up the standard curve of the know standard to obtain the estimated actual concentration.

According to a third aspect of the present disclosure, the estimated actual concentration is determined within 5 seconds of gas exposure. The alarm may be triggered to signal the estimated actual concentration is above the predetermined threshold level with less than 5 seconds of gas exposure.

According to a third aspect of the present disclosure, the step of determining the estimated actual concentration of the detected gas includes taking into account a modifier coefficient identified from the determined rate of change.

According to a third aspect of the present disclosure, the step of determining the estimated actual concentration of the detected gas includes taking into account a time variance due to a sensitivity of a sensor of the gas detector.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 shows an embodiment of a gas detection system, having a detector that includes a housing, a sensor, and an output display;

FIG. 2 shows the gas detection system of FIG. 1 with part of the housing of the detector removed to show the internal circuitry, and showing the gas detection system includes an analog-to-digital converter (ADC) connected to the sensor and a controller of the gas detections system;

FIG. 3 shows example steps of a method of estimating an actual concentration of a gas detected by the gas detection system of FIG. 1 by comparing a determined rate of change of concentration of the gas detected by the gas detection system to a known standard;

FIG. 4 shows example steps of a subprocess of the method of FIG. 3 where the known standard is derived from normalized characteristic curves of known gas concentrations;

FIG. 5 shows example steps of a subprocess of the method of FIG. 3 where a standard curve is derived from the known standard characteristic curves to compare with the measured rate of change of concentration of the detected gas to calculate an estimated actual concentration of the detected gas;

FIG. 6 shows example steps of a subprocess of the method of FIG. 3 where additional modifiers and coefficients are factored into the calculation used to determine an estimated actual concentration of the detected gas to account for various sensor specifications and increase the accuracy of the estimate;

FIG. 7 shows example steps of a subprocess of the method of FIG. 3 where the estimated actual gas concentration is used to trigger a warning or other external output on a detector of the gas detection system for use as an early warning or in a bump test situation;

FIG. 8 shows an example of normalized characteristic curves of a known gas concentration and known sensor using 2000 counts per parts per million (ppm), showing examples of characteristic curves used as the known standard for the gas detection system of FIG. 1;

FIG. 9 shows an example of an identified rate zone, over which a rate of change of concentration of the detected gas is calculated, on the 50 ppm characteristic curve of FIG. 8;

FIG. 10 shows an example of a smooth and normalized standard curve derived from the characteristic curve of FIG. 9;

FIG. 11 shows a detailed view of a portion of the standard curve of FIG. 10, showing the location of the rate zone at a point of the standard curve where gas is evident and is suitable to determined rate of change of the detected gas;

FIG. 12 shows a rate modifier curve for a 50 ppm concentration gas based on the derivative of the inverse of the characteristic curve of FIG. 11;

FIG. 13 shows examples of calculated modifier coefficients for a 50 ppm gas;

FIG. 14 shows an example of the time variance between the different characteristic curves of sensors with various sensitivities;

FIG. 15 shows a detailed view of a portion of the characteristic curves of FIG. 14, further illustrating the time variance and time variant shift of each sensor;

FIG. 16 shows example of calculated changes in rate zone measurements due to the time variance illustrated in FIG. 15;

FIG. 17 shows the difference in response speed provided by the early gas detection method of FIG. 3 utilized by the gas detection system of FIG. 1 compared to the T90 response;

FIG. 18 shows example estimated actual concentrations of a known gas calculated using the early gas detection method of FIG. 3 for relatively high sensitivity sensors;

FIG. 19 shows example estimated actual concentrations of a known gas calculated using the early gas detection method of FIG. 3 for relatively low sensitivity sensors;

FIG. 20 shows a summary of example estimated actual concentrations of a known gas calculated using the early gas detection method of FIG. 3;

FIG. 21 shows example calculations of the time savings provided by using the early gas detection method of FIG. 3 compared to the T90 response; and

FIG. 22 shows an example of estimated actual concentrations of a relatively high concentration known gas calculated using the early gas detection method of FIG. 3 for relatively low sensitivity sensors.

### DETAILED DESCRIPTION

While the disclosure may be susceptible to embodiment in different forms, there is shown in the drawings, and herein will be described in detail, specific embodiments with the understanding that the present disclosure is to be considered an exemplification of the principles of the disclosure, and is not intended to limit the disclosure to that as illustrated and described herein. Therefore, unless otherwise noted, features disclosed herein may be combined to form additional combinations that were not otherwise shown for purposes of brevity. It will be further appreciated that in some embodiments, one or more elements illustrated by way of example in a drawing(s) may be eliminated and/or substituted with alternative elements within the scope of the disclosure.

Gas detectors and sensors are used to detect harmful gases, such as carbon monoxide, in a variety of environments. In particular, gas detectors may be used to detect harmful gases in work environments such as oil refineries, gas pipelines, and other facilities. Sensor response is indicated by an industry standard T90 value, which is the time the sensor takes to reach 90% of the gas exposure level. Typical detectors have T90 response times from 15 to 30 seconds. Gas detectors have alarm setpoints in ppm (parts per million) of gas concentration for the purpose of warning the user. When the setpoint is reached the device alarms accordingly. The time it takes to get to the warning is proportional to the characteristic T90 response of the electrochemical sensor.

The method and systems describe herein utilize a sample coefficient to estimate gas concentration in the early stages of exposure, maximizing the ability for a user to move away from danger much quicker than is currently accepted. The method of obtaining the early sample coefficient is possible by the systems and method described herein, which utilize normalized parameters such as analog-to-digital converter (ADC) counts measured, calculated parts per million (ppm), and percent maximum concentration of a gas.

As will be explained below, in the illustrative embodiments, the sample coefficient is utilized to estimate a gas exposure level in, for example, 3-4 seconds of gas application to a gas sensor 120 of a gas detection system 100. The gas detection system 100 maintains the linearity of the sensor 120 throughout circuitry 170 and calculations within a code stored within a memory 154 of a controller 150 of the gas detection system 100. The consistent linearity throughout allows normalization between detectors and at all the exposure levels in the gas detection system's 100 concentration range. The gas detection system 100 utilizes an early gas detection method 300 used to calculate the coefficient. The method 300 is based on creating characteristic cubic polynomial curves and the law of tangents. Coefficient Standardization is from the standard curve developed from testing with a calibration gas which represents the low end of the operating concentration range and sensitivity of one.

The time savings provided by the early gas detection method 300 and gas detection system 100 describe herein is particularly valuable as a safety feature in higher concentration events. Using the early gas detection method 300 to projecting or estimate an actual concentration of a detected gas results in a relatively early alarm, allowing users to more quickly identify dangerous levels of gas exposure. Industrial gas exposures happen in many ways with most being unforeseen. The health of the user during a gas exposure is dependent upon fast reaction when gas is detected. The earlier a harmful concentration is detected the better the chances of escape before harmful effects.

The benefits of detecting higher concentrations of gas in, in the illustrative embodiments, 3-4 seconds and warning the user in a fraction of the time that it normally takes is valuable from a safety perspective. The linear relationship between the concentrations from the system of the normalization characteristic curves described herein provides a way to project or estimate the actual concentration of the detected gas to alarm concentration levels. The rate zone utilizes in the method 300, as will describe in more detail below, provides an early look at where a cubic polynomial curve representative of the concentration of the detected gas is going to maximize. The system normalization allows for comparing concentrations with the rate zone to calculate an estimated actual concentration of the detected gas.

Without the early alarm method 300, the T90 response will determine how fast a gas concentration at alarm threshold would be detected. In the illustrative embodiments, the method 300 described herein and the gas detection system 100 may supply a warning in 3-4 seconds from exposure of the gas to the gas sensor 120, compared to 15-30 seconds in some situations with other typical solutions. In other embodiments, the method 300 and the gas detection system 100 may supply a warning in less than 3-4 seconds from exposure of the gas to the gas sensor 120, or more than 3-4 seconds from exposure of the gas to the gas sensor 120. Regular intervals of ADC readings will continue after the early warning is initiated to reinforce that the estimated actual gas concentration is true and to return device output to normal operation when the gas subsides.

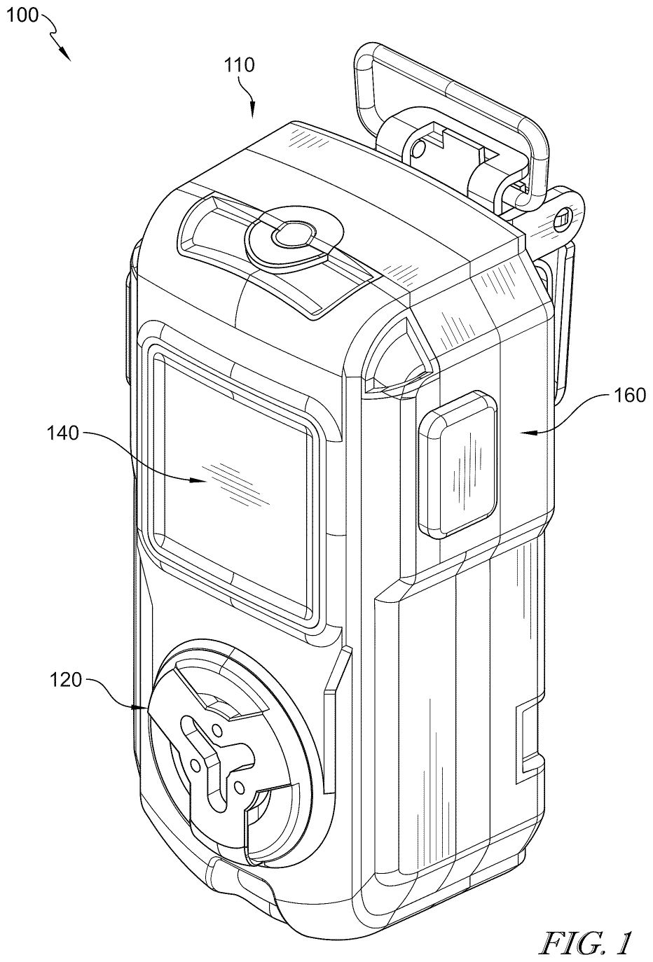

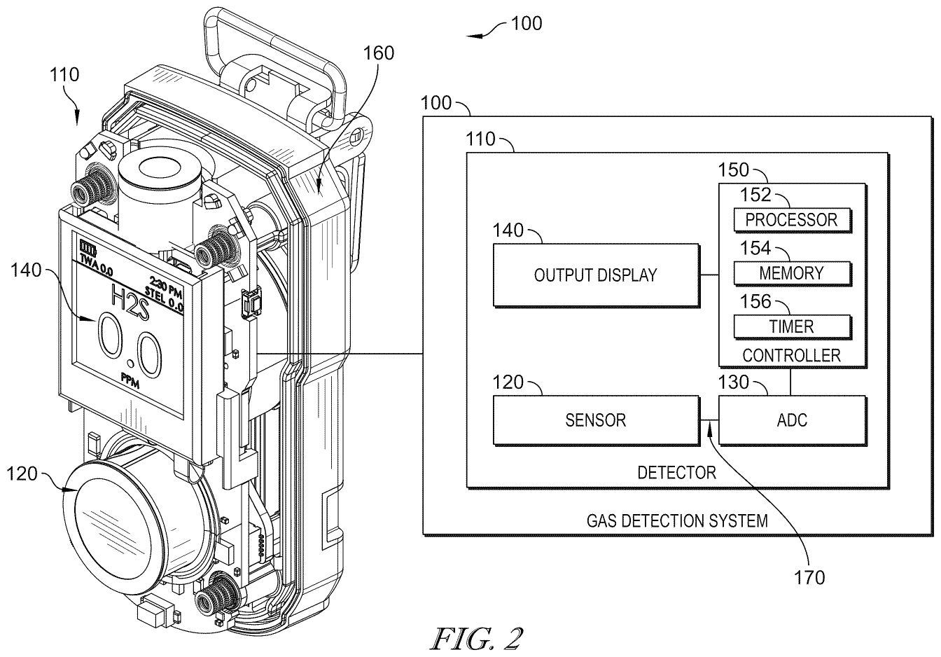

Turning now to the illustrative embodiment, the gas detection system 100 of the present disclosure, shown in FIG. 1, is operable to warn users of the presence of harmful gas. In the illustrative embodiments, the gas detection system 100 includes a gas detector 110 having a gas sensor 120, an analog-to-digital converter (ADC) 130, an output display 140, a controller 150 having a processor 152 and memory 154, and a housing 160, as shown in FIGS. 1-2. The gas detection system 100 includes circuitry 170 connecting the components. In some embodiments, the gas detection system 100 includes an audible alarm, a flashing light, and/or a vibrating motor in addition to or in place of the display 140 to notify users a harmful gas is present. In the illustrative embodiment, the detector 110 is shown as a PROTECTOR Handheld Intrinsically Safe and IP67-Rated Gas Detector by Molex. Additional details of the PROTECTOR detector 110 may be found in the documents entitled “Operating Manual” and “Quick Start Manual,” the contents of which are incorporated by reference herein in their entirety in the Appendix. In other embodiments, the gas detection system 100 is used with a variety of detectors 110 and/or sensors 120.

In some embodiments, the gas detection system 100 includes multiple detectors 110 connected to form a network of detectors 100. In other embodiments, the gas detection system 100 may only include one detector 110. In some embodiments, the network of detectors 110 may all be connected to a shared cloud storage and/or cloud application 180 with online storage and data processing capabilities, as shown in FIG. 2. The detectors 110 may be connected to the cloud application 180 via Bluetooth. A user may be able to access the cloud application 180 via a phone application and/or a computer application. A user may monitor real time data and/or access historical measurements from the detectors 110 via the application 180. Additionally or alternatively, a user may view and/or control detector 110 settings and operations. For example, bump tests, calibration, and/or system and firmware updates may be initiated and controlled via the application 180.

In the illustrative embodiments, the gas detection system 100 is operable to utilize the early gas detection method 300 to provide an early warning of an impending gas exposure. In the illustrative embodiments, the early warning is provided within 3 to 4 seconds of the sensor 120 being exposed to a gas. The method 300 allows the gas detection system 100 to provide an early warning for all concentrations of gas within the detection system's 100 design range. In some embodiments, the projection method 300 is applicable for any gasses and all concentration ranges. In the illustrative embodiment, the user is able to choose whether they want the gas detection system 100 to operate using the early gas detection method 300, or using the typical T90 response described above.

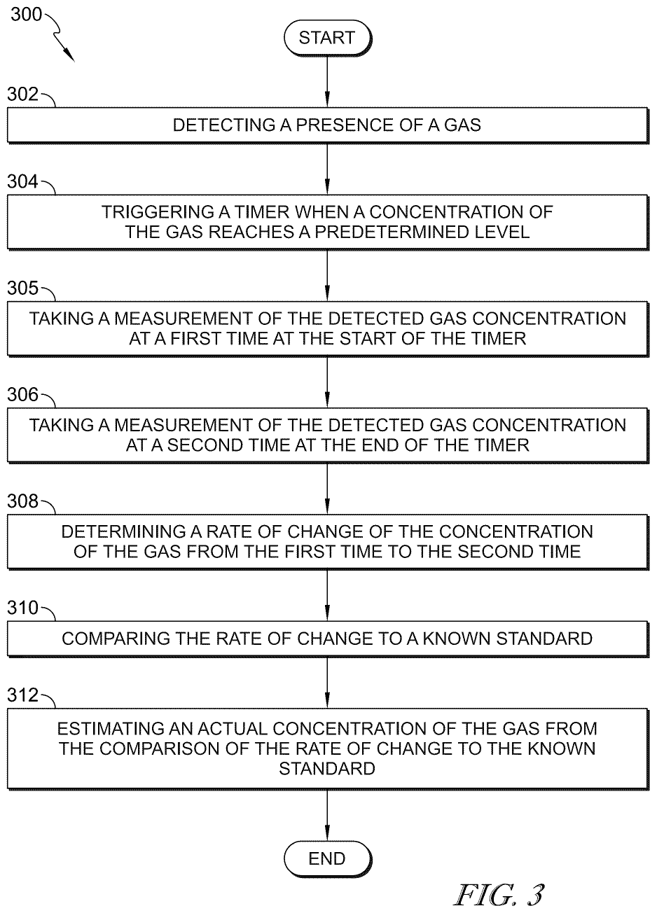

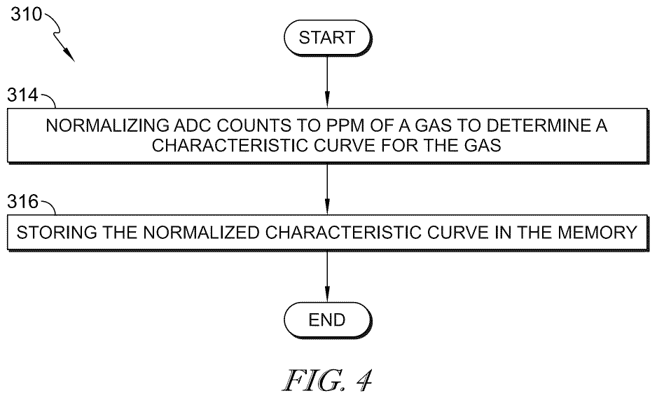

Turning to FIGS. 3-7, the gas detection system 100 is capable of calculating an estimated actual concentration of gas detected by the sensor 120. FIGS. 3-7 illustrate a process of operating the system 100 in accordance with the early gas detection method 300. The method 300 shown in FIGS. 3-7 may have fewer or additional steps, may have repeated steps, and/or may be performed in a different order than shown. Any of the steps illustrated in FIGS. 3-7 may be performed with any of the other steps shown in FIGS. 3-7. For example, one or more steps of the method 300 shown in FIGS. 3-7 may be performed as a subprocess as a part of a larger method 300 with other steps shown in FIGS. 3-7. For example, the steps shown in FIGS. 4-7 may, in some embodiments, be optional subprocess of one or more of the steps shown in FIG. 3.

As will be described in further detail below, the method 300 comprises a detecting step 302, which includes detecting the presence of a gas with the sensor 120. The method 300 comprises a triggering or initiating step 304, which includes triggering a timer when a concentration of the detected gas at the sensor 120 is determined to reach a predetermined level or threshold. The method 300 comprises a measuring step 305, which includes taking a first measurement, or a first reading, of the concentration of the detected gas at the sensor 120 at a first time. In the illustrative embodied, the first time corresponds to the time the timer is started. The method 300 comprises a measuring step 305, which includes taking a second measurement, or a second reading, of the concentration of the detected gas at the sessor 120 at a second time. In the illustrative embodiment, the second time corresponding with the end of the timer.

The method 300 comprises a determining step 308, which includes determining a rate of change of the concentration of the detected gas from the first time to the second time. The method 300 includes a comparing step 310, which includes comparing the determined rate of change to a known standard. In the illustrative embodiments, the known standard corresponds to normalized characteristic curves as will be described below. The method 300 includes an estimating step 312, which includes estimating or calculating an estimated actual concentration of the detected gas from the comparison of the determined rate of change to the known standard.

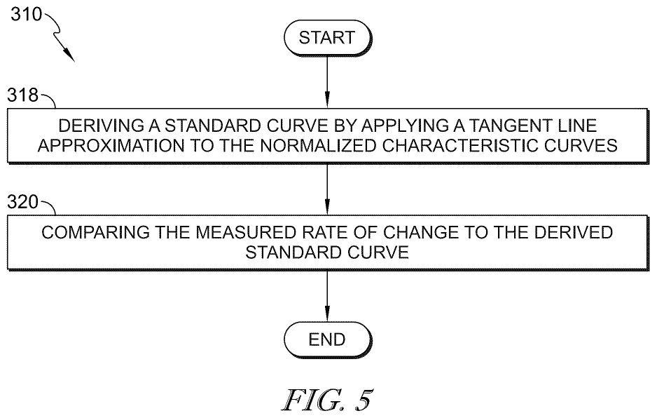

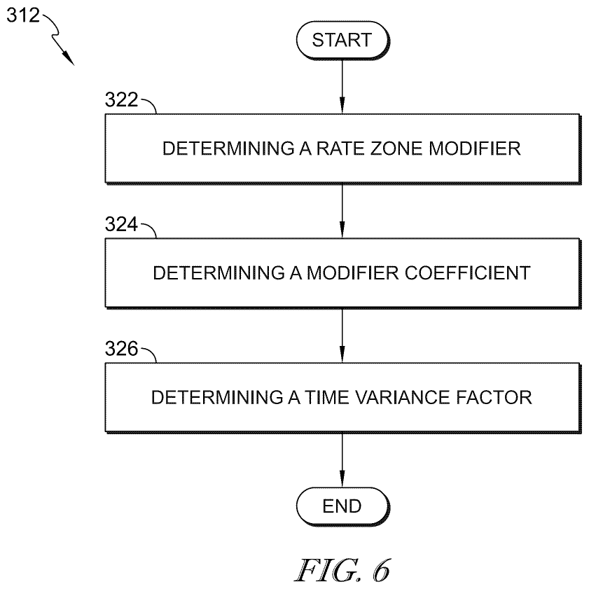

In some embodiments, as shown in FIG. 4, the method 300 includes a subprocess where the comparing step 310 further includes a normalizing step 314 and a storing 316 step. The normalizing step 314 includes normalizing ADC counts to ppm of a gas to determine characteristic curves for a known gas. The storing step 316 includes storing the normalized curves in the memory of the gas detection system 100. In some embodiments, as shown in FIG. 5, the method 300 includes a subprocess where the comparing step 310 further includes a deriving step 318 and a comparing 320 step. The deriving step 318 includes deriving a standard curve from the normalized characteristic curve. The comparing step 320 includes comparing the measured rate of change to the derived standard curve. In some embodiments, as shown in FIG. 6, the method 300 includes a subprocess where the estimating step 312 further includes one or more additional determining steps 322, 324, 326. A determining step 322 may include determining a rate zone modifier to apply to the estimation calculation. A determining step 324 may include determining a modifier coefficient to apply to the estimation calculation. A determining step 326 may include determining a time variance factor to apply to the estimation calculation.

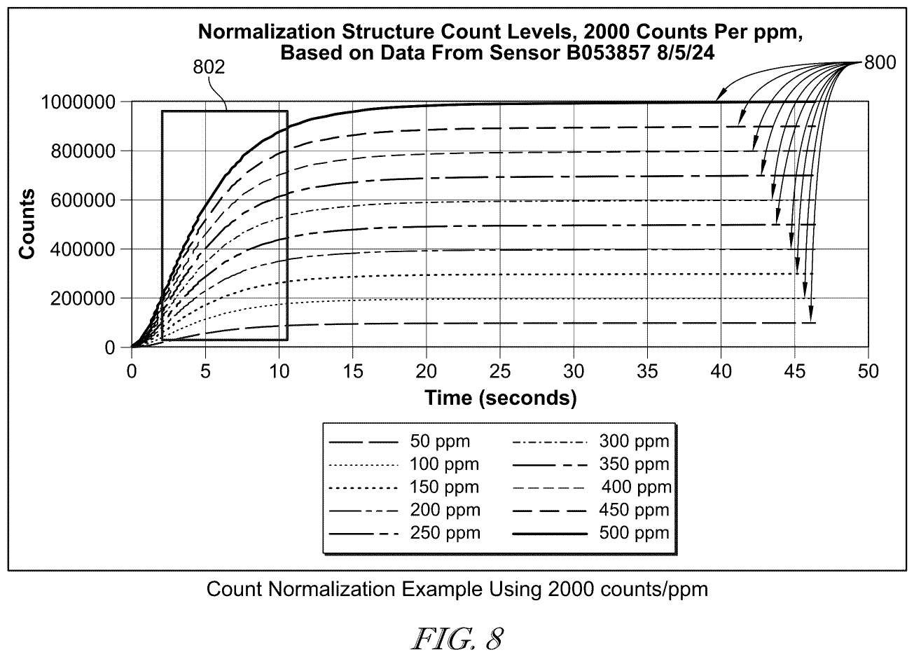

Turning back to FIG. 4, the method 300, in normalizing step 314, normalizes measured ADC counts to a value per parts per million (ppm) suitable for the desired gas types and ranges. In the storing step 316, the normalized data is stored in the memory 154 of the gas detection system 100. The counts are normalized using a sensitivity of the sensor 120 in the detection system 100. Sensitivity for sensors is measured as part of the sensor manufacturing process and is stored in the memory 154 for computations. The normalization step 314 using sensor sensitivity and counts per ppm provides predictable characteristic curves 800 for use by the gas detection system 100, shown in FIG. 8.

FIG. 8 shows the relationship between the characteristic curves 800 for various concentrations of gases. In the illustrative example shown in FIG. 8, the characteristic curves 800 are represented by the equation:

$$Counts = ( 2 \; 000 ) ( gas concentration )$$

$$gas concentration = counts / 200 \; 0 .$$

This relationship allows each characteristic curve 800 to be uniform in magnitude of applied gas ppm. The uniform characteristic curves 800 allow for generic functions for describing the sensor 120 output levels relative to gas concentrations, as will be described in more detail below.

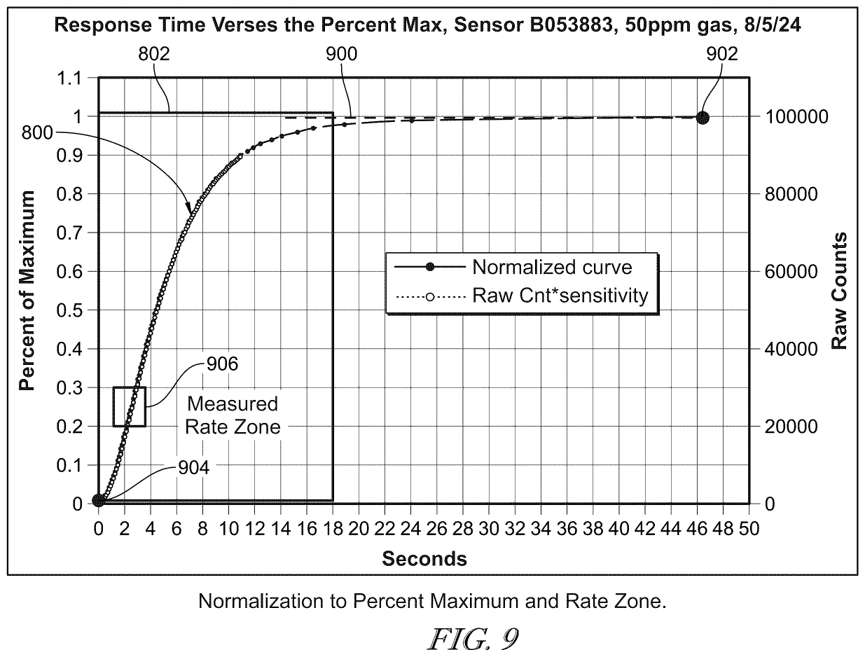

An example characteristic curve 800 for a 50 ppm gas is shown in FIG. 9. The rising portion 802 of the characterization curve 800 before the asymptotic knee 900 of the curve 800 can be described as a cubic polynomial function. Taking a second derivative of the cubic polynomial function represents the deceleration of the curve. The second derivative of the of the characterization curve 800 before the asymptotic knee 900 represents a linear deceleration. In the illustrative embodiments, the deceleration is the amount of decrease in response rise until the curve goes asymptotic at the highest magnitude 902. In other words, for the characterization curve 800 before the asymptotic knee 900, the rate of change decreases in a linear fashion as the cubic polynomial function goes from near zero 904 to maximum 902. The method 300 utilizes a measured rate of change in concentration of a detected gas near the beginning of the response rise, as determined in step 308, to project a path up the normalized curve 800 and estimate the expected concentration of the gas.

The method 300 utilizes an early gas exposure function, which operates in the deceleration zone 906 of the curve 800. This zone 906, also known as the rate zone, illustrated in FIG. 9, is selected to be in a portion of the curve 800 with a relatively higher expected rate per second change. The rate zone 906 is also selected to be far enough up the curve 800 to ensure gas flow to the sensor 120 is established, the detector 110 is reading concentration, and the readings are increasing. In some embodiments, the rate zone is selected to meet specific system characteristics and specific applications.

In the illustrative embodiments, one the sensor 120 detects, in detecting step 302, a presence of a gas, the method 300 measures 305 the ADC counts of the detector 110 at a first time, when the counts are in the region around 20% of the exposed gas concentration. The method 300 also measures 306 the ADC counts at a second time, a second after the first time in the illustrative embodiment, around 30% of the exposed gas concentration in the illustrative embodiments. The method 300 includes determining 308 a difference, or delta, of the two count measurements to represent the measured rate of change the curve 800 over one second.

In some embodiments, the gas detection system 100 employs a timer 156 that is set and controlled by the controller 150 such that the ADC counts are measured and/or stored at the beginning and end of the timer 156. In the illustrative embodiment, the timer 156 is set for a 1 second period. In the illustrative embodiment, the method 300 includes the controller 159 activating or triggering, in triggering step 304, the timer 156 when the detected gas reaches a predetermined level. In the illustrative embodiments, the timer is triggered when the measured ADC counts reach a predetermined level that corresponds to 20% or 0.20 percent of the maximum gas concentration for the desired gas. In some embodiments, this predetermined level of ADC counts is determined from the corresponding characteristic curve 800 for the desired gas and concentration. In the illustrative examples for a 50 ppm gas, the ideal rate zone start corresponding to 20% is 20,000 ADC counts for a 2000/ppm system.

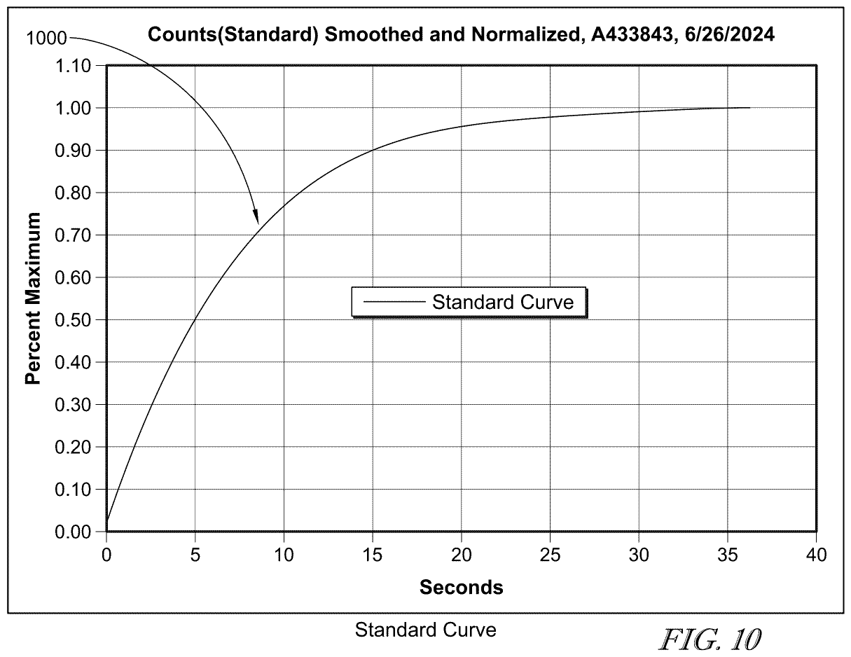

In the illustrative embodiments, the measured rate of change in the rate zone 906, over the duration of the timer, represents a tangent of a standard curve 1000, as shown in FIG. 10. The measured rate is indexed to a constant area on the normalized characteristic curve 800, as shown in FIG. 10. In the illustrative embodiment, in the deriving step 318 the standard curve of the detected gas is derived by applying a tangent line approximation to the normalized characteristic curves stored in the memory 154 of the system 100 to obtain, in the comparing step 310, comparative values for expected, actual counts of the detected gas. From the measured rate, a calculation similar to a tangent line approximation is used in step 312 to estimate the actual concentration value by projecting up the standard curve. The standard curve can be described as a cubic polynomial function in a range up to T90, which encompasses the region of interest for triggering a gas detection alarm.

In the illustrative example shown in FIG. 10, the test data used to develop the standard curve of a detected gas is from the sensor A433843, a typical sensor that exhibits a T90 value of 15 seconds and a sensitivity of 1. The standard represents a T90 in the middle of the typical expected range of T90s and sensitivity of 1 is what the system 100 is normalized to in the illustrative embodiment. The method 300 then utilizes the standard curve for calculating the estimated actual concentration of the detected gas in estimating step 312.

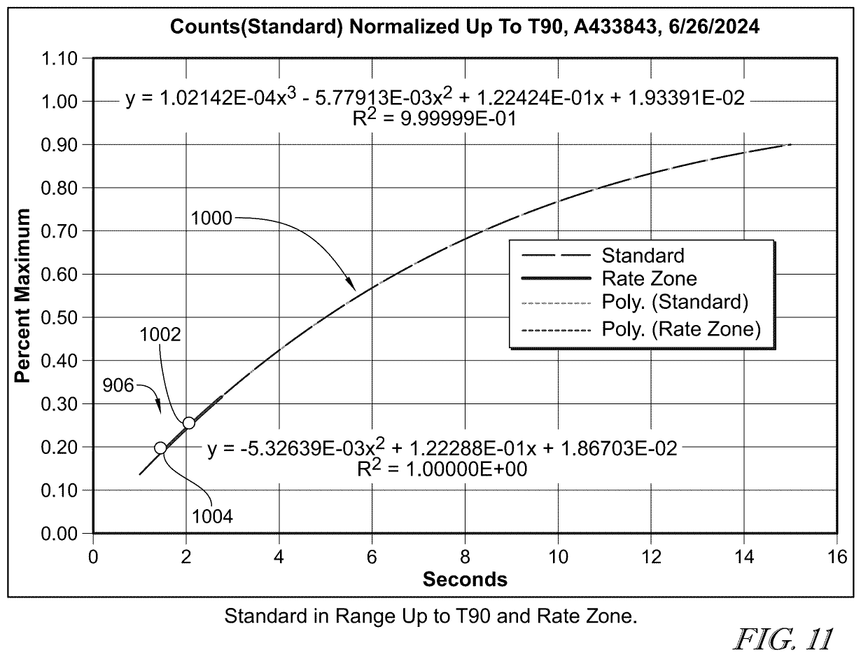

In the illustrated embodiment using the example data and sensor, to determine the estimated actual concentration of the gas, the method 300 includes comparing steps 310 and 320 for comparing the measured ADC counts within the rate zone 906 to the values derived in the standard curve, as shown in FIG. 11. In the illustrated example, the rate zone 906 in the example in FIG. 11 spans over the 20% to 30% along the standard curve 1000 and the standard curve 1000 has a tangent near the 25% concentration of gas applied. To estimate the expected actual ADC counts of the detected gas, the method 300 includes first taking the derivative of the polynomial fit in the rate zone 906. Calculations for the example of the gas and sensor shown in FIG. 11 for this process are shown below:

$$Rate Zone Polynominal : Y = - 0.0053 \; 2639 {x}^{2} + 0.12 \; 2288 x + 0 \text{.0186703}$$

$$Derivative Rate Zone Polynominal : Y ’ = - 0.01065278 x + \text{.122288}$$

$$Zone Mid - Point at 0.25 : Y ’ = 0.11815605$$

$$Percentage of Maximum Counts : Expected counts = ( 0.11815605 ) ( 100 ) = 11.816$$

$$Multiplier = 100 / 11.816 = 8.4631$$

As the calculations in the example above are done in percent of maximum value to enable projection up the characteristic curve 800, a means of applying a multiplier to estimate the projected or estimated actual concentration of the gas is required. In the above example calculations for a 50 ppm characteristic curve 800 targeting a rate zone near 0.25 percent maximum, a multiplier may be obtained directly using the derivative of the rate zone cubic polynomial, as shown above. In some embodiments, the gas detection system 100 does not act as a data acquisition system, and a time base accurate enough to start the rate zone exactly at the desired 0.20 maximum desired is not used. In such embodiments, the method includes in determining step 322, determining a rate zone modifier to use such that the universal algorithm is useful for all gas exposure concentrations.

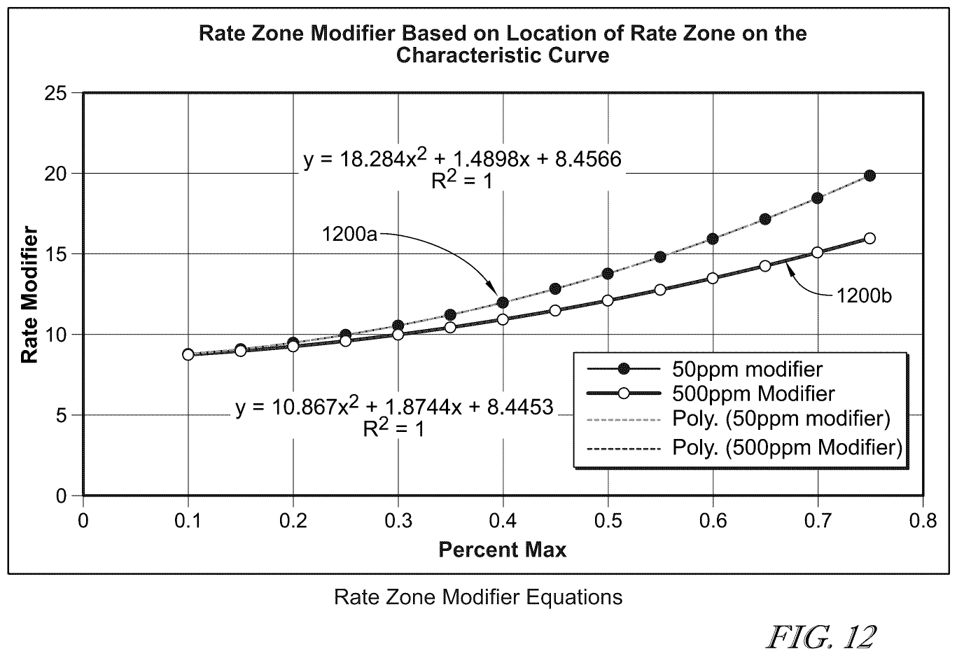

For example, in an illustrative test, the characteristic curves 800 for a 50 ppm gas and a 500 ppm gas were analyzed to verify the curves are the same shape and multiplier coefficients were calculated for both. The resulting data suggests that, in some embodiments, the gas detection system 100 may inherently overshoot the ideal rate zone start of 20,000 counts for a 2000/ppm system, and higher concentrations may reach 20,000 counts before being at 0.20 percent of maximum concentration. In some embodiments, to compensate for low and high concentrations, a sub-algorithm modeling the position of the rate zone measurements on a derivative of the inverted rate zone percent over time is used. As seen in FIG. 12, example rate modifier curves 1200a, 1200b for two different sensors at 50 ppm are shown. The modifier curves 1200a, 1200b are based on the derivative of the inverse of the characteristic curve of FIG. 11.

In some embodiments, the method includes in determining step 324, determining a modifier coefficient to be used with the rate zone modifier to give a multiplier. The modifier coefficient is derived from measured data in the rate zone 906. In some embodiments, the center of the measured zone 906 is not easily ascertained without resolution comparable to a data acquisition, so a method to approximate a location may be employed.

In the illustrative embodiments, the closest measured value to accurately approximate the rate zone 906 location is the second counts reading at the second time, or the counts reading at the end of rate zone 906 measurement. Shifting half of the delta backward approximates the center or tangent of the rate zone 906. In the illustrative example, the calculations are shown to have a rate zone 906 center 1002 at 0.25 percent maximum concentration and a rate zone 906 start 1004 at 0.20 percent maximum concentration. In the illustrative example, with a 50 ppm concentration gas, the start 1004 corresponds to 2 seconds after gas exposure, the center 1002 corresponds to 2.5 seconds after gas exposure, and an end of the rate zone at 0.3 percent maximum concentration corresponds to 3 seconds after gas exposure.

Accordingly, for the illustrative examples, the modifier coefficient is adjusted to make the 50 ppm range with no additional modifier scaling or equal to 1 and to normalize higher concentration's modifier coefficient to the 50 ppm range. In the illustrative embodiment, the −0.25 term in the modifier coefficient equation sets a rate modifier to approximately 1 at 50 ppm and allows scaling for higher ppm concentrations from that anchor point. The modifier coefficient for the describe example is modelled as follows:

$$Modifier Coefficient : ( second reading - Delta / 2 ) - ( Start - 20000 ) ) / 100000 ) - 0.25 - ( 0.024 / sensitivity )$$

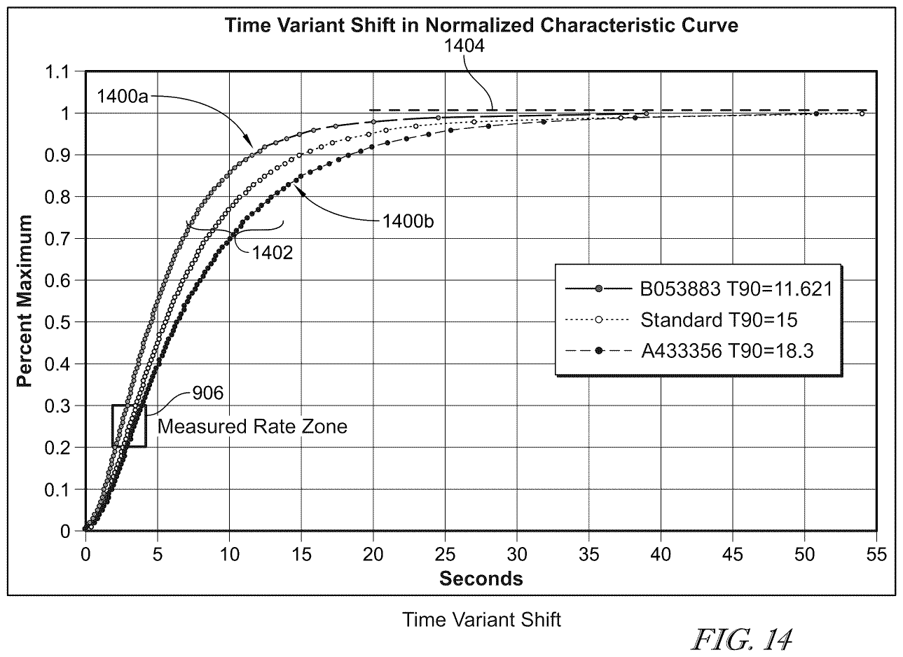

The last term of the above example equation, (0.024/sensitivity) will be discussed in further detail below. The time variance in T90 characteristic times causes a change in the rate of change moving up to the steady state and is also present in the rate measurement zone. As show in FIG. 14, a fast sensor will read more counts in a second compared to a slower sensor. In the illustrative example, the gas detection system 100 is not trying to precisely model this time variance. Instead, the system 100 calculates an estimated actual concentration of the gas by projecting up the normalization curve 800 to 100% maximum gas concentration. Accordingly, in some embodiments, the time variant shift depicted in FIG. 14 is addressed.

As can be seen in the illustrated example shown in FIG. 14, the rise to the normalized maximum is dependent upon the response characteristics of each specific sensor. Depicted in the example in FIG. 14 are characteristic curves 1400a, 1400b for a B053883 sensor, a relatively fast responding sensor, and an A433356 sensor, a relatively slow sensor. The time variance 1402 is far more prominent as the characteristic curve approaches the asymptotic region 1404 compared to the rate measuring zone 906. The variance is significant since the idea of early detection of gas exposure projects past the slow rising asymptotic regions to provide an exposure value faster. The differences between the curves 1400a, 1400b increase until the asymptotic region 1402 and then converge. This illustrates another reason to set the rate zone earlier in the curve, before the variance increases.

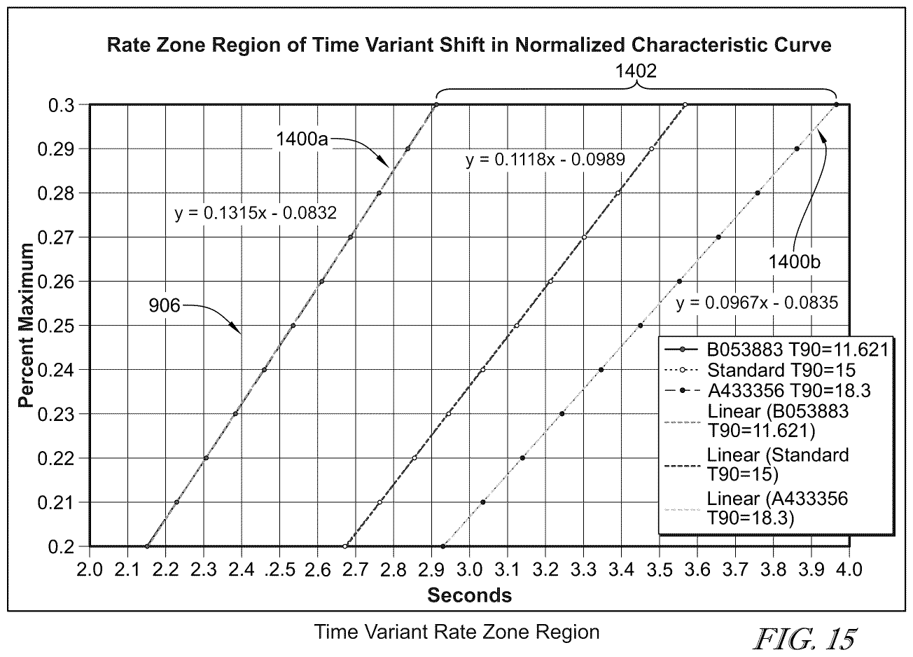

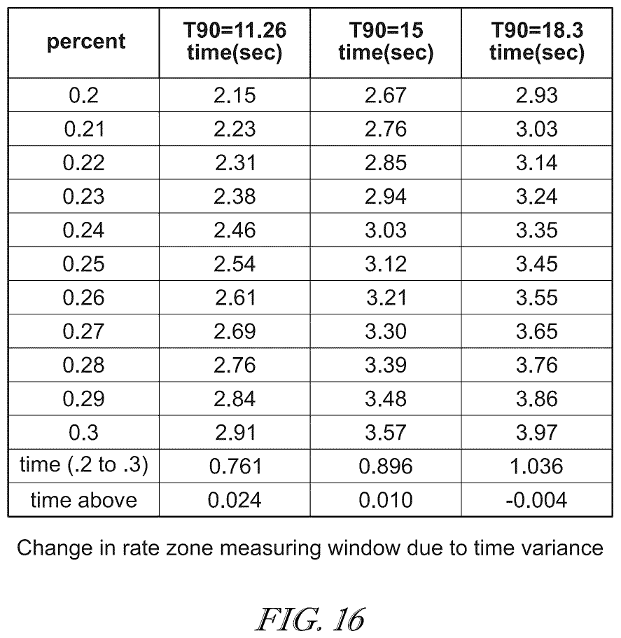

FIG. 15 illustrates a zoomed in view of the rate zone 906 of the time variant shift in FIG. 14, showing how the respective T90 response rates of the various sensors are contributing before the rate zone measurement, causing the faster responding sensors to reach the rate zone 906 quicker. The gas detection system 100 in the illustrative embodiments initiates 304 the rate zone 906 by ADC counts and not by time. Due to this, the various response rates do not significantly affect the method 300 used by the gas detection system 100. Although faster sensors will have more ADC counts in rate zone 906 by the nature of a steeper slope through the rate zone, the difference isn't significant. The additional ADC counts for relatively faster sensors due to variance is not significant at lower gas concentrations as the error is about +/−0.5 percent. However, the error increases with increased concentration range and therefore will be addressed. FIG. 16 shows examples of various rate zone measuring windows due to time variance.

In the illustrative embodiments, the gas detection system 100 and method 300 addressed the time variance in the rate zone 906 with a time variance factor using sensitivity as a speed variable, the time variance factor, as determined in the determining step 326, is added to the above modifier coefficient equation. This additional term to the modifier coefficient, shown as the last term in the above equation, has very little impact at low concentration but the errors scale with increase in concentration.

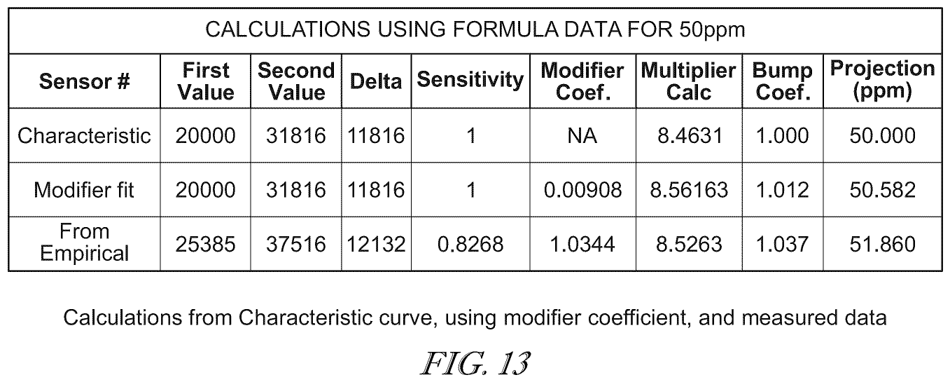

Turning back to the modifier coefficient, in the illustrative embodiments, for a projected concentration, in the determining step 324 the modifier coefficient is calculated from the measured ADC counts and the specific sensor sensitivity. The modifier coefficient is utilized to calculate a modifier to be used as a multiplier on the delta to calculate 312 the estimated actual concentration of the detected gas. In some embodiments, the multiplier is used to determine a bump coefficient which is used as comparisons to previous bump coefficients with known gas applied. In the illustrated embodiment, the modifier coefficient is used with the rate modifier equation shown for 50 ppm example shown in FIG. 12. Example calculations of the modifier coefficient on the characteristic curve 800 are shown in FIG. 13 for a 50 ppm gas exposure. As shown, the results are close to the ideal projection goal. The equation for the multiple is shown below, where x is the modifier coefficient.

$$Multiplier = 1 \; 8284 {x}^{2} + 1.4898 x + 8 \text{.5466}$$

$$Projected ppm = ( measured delta ) ( multiplier ) ( 50 )$$

$$Bump Coefficient for 2000 / ppm = ( measured delta ) ( multiplier ) / 100000$$

With these calculations, the gas detection system 100 and early gas detection method 300 determine 312 the projected estimated actual gas exposure concentration of the detected gas. The step 314 of normalization using sensitivity and counts/ppm set parameters and known expected values for each detector and between detectors. Measuring the rate of change in the same rate area 906 of the cubic polynomial characteristic curve 800 provides a means of projecting the estimated gas exposure determined by the structured normalization.

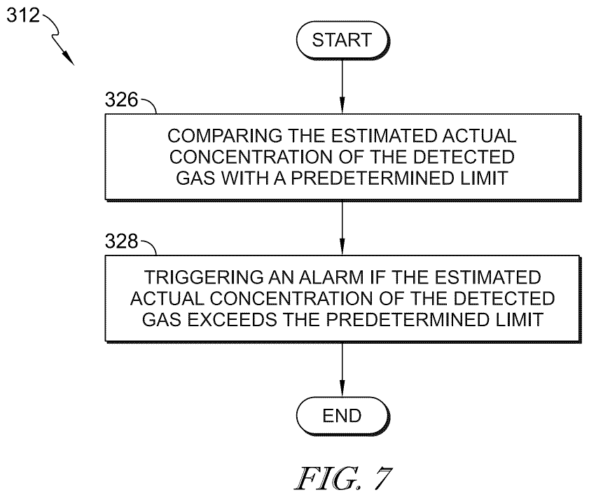

In some embodiments, as shown in FIG. 7, the method 300 includes a subprocess where the estimating step 312 further includes a comparing step 326 and a triggering step 328. The comparing step 326 comparing the estimated actual concentration of the detected gas with a predetermined limit. In the illustrative embodiments, the predetermined limit corresponds to a gas alarm limit of the gas detection system 100. The triggering step 328 includes triggering an alarm of the gas detection system 100 if the estimated actual concentration of the detected gas exceeds the predetermined limit. In the illustrative embodiment, the alarm may be an image or warning displayed on the output screen 140, and or the triggering of other visual, audio, or vibration alarms.

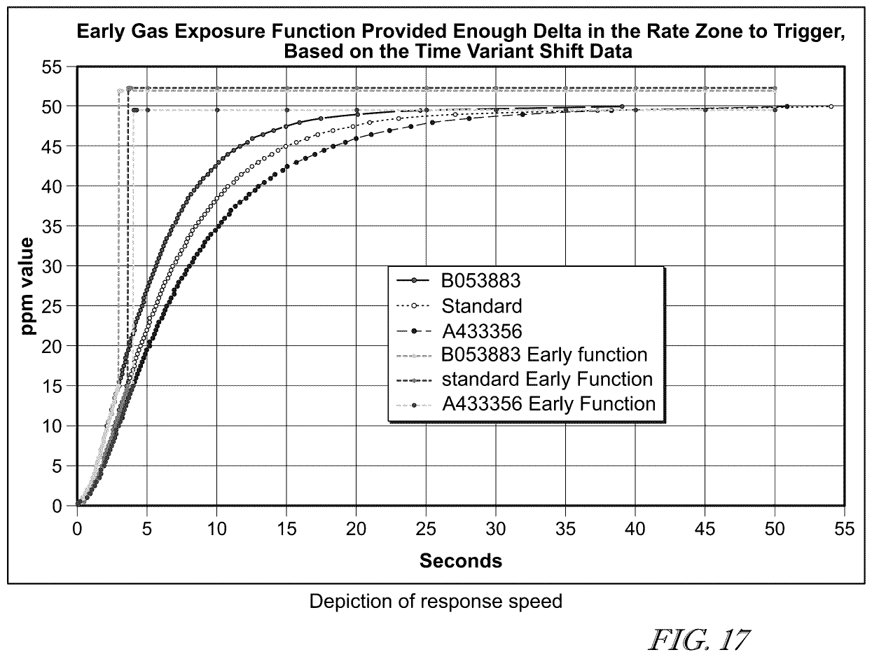

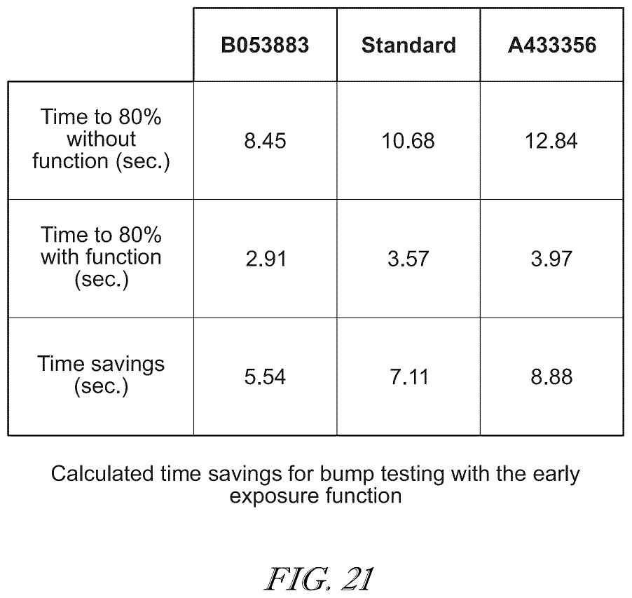

The gas detection system 100 and the early gas detection method 300 may be used for detecting a calibration gas very quickly, and similarly be extended to higher concentration gas exposures. The described method 300 may be utilized in bump testing the sensor 120 and/or the gas detection system 100. A typical bump test will introduce a calibration gas until the detector reads 80% of the calibration gas concentration. A sensor near the parameters of the standard will typically take 10.68 seconds to reach 80% calibration gas. In contrast, the early gas detection method 300 is able to indicate the gas will reach the same threshold in 3-4 seconds. The same logic as describe above is utilized for bump testing so the bump criteria for testing the sensor 120 may be set at the lower end of the concentration range, allowing the function to be used for gas bumps and for scaling up to higher concentrations. In the illustrative embodiments, the faster indication may save 5.54 to 8.88 seconds on a low-level alarm threshold. Faster bump testing can consequently save gas and money, as less gas is needed for the threshold to be determined, and less time is needed to test each sensor. An example of the benefit using the early gas exposure function can be seen in FIGS. 17 and 21.

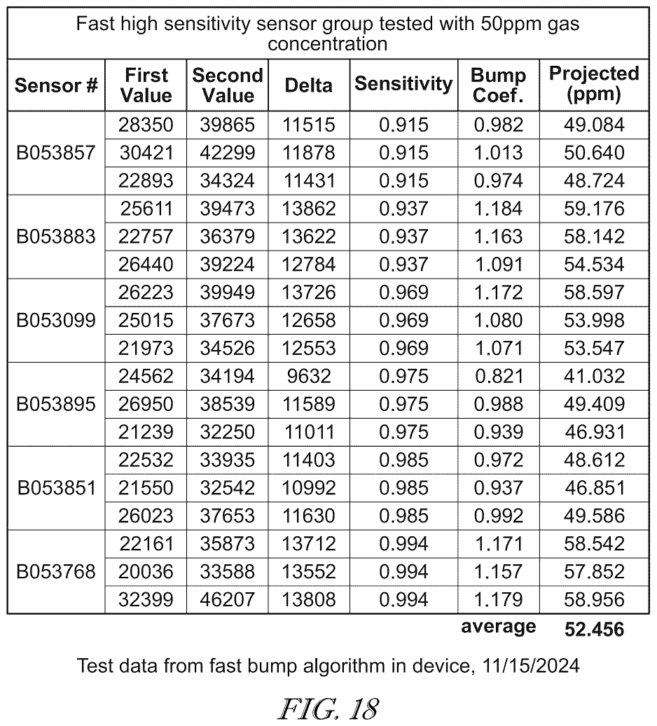

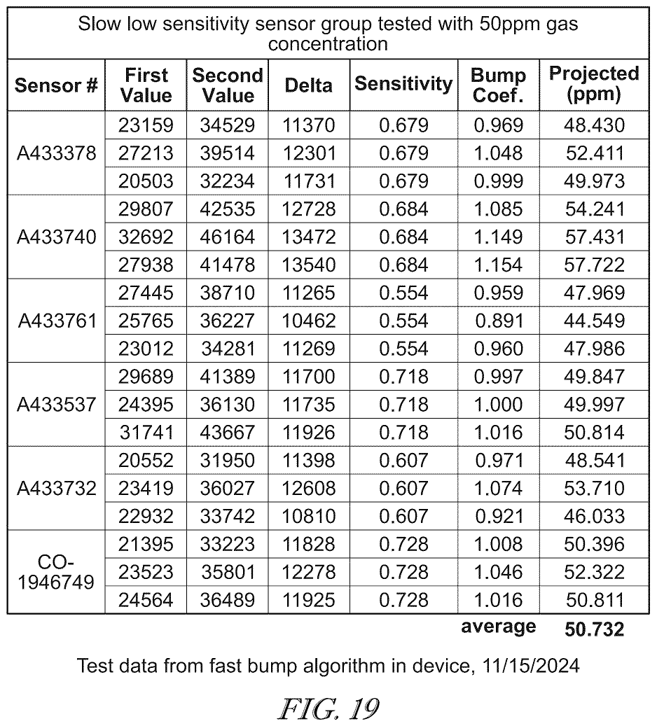

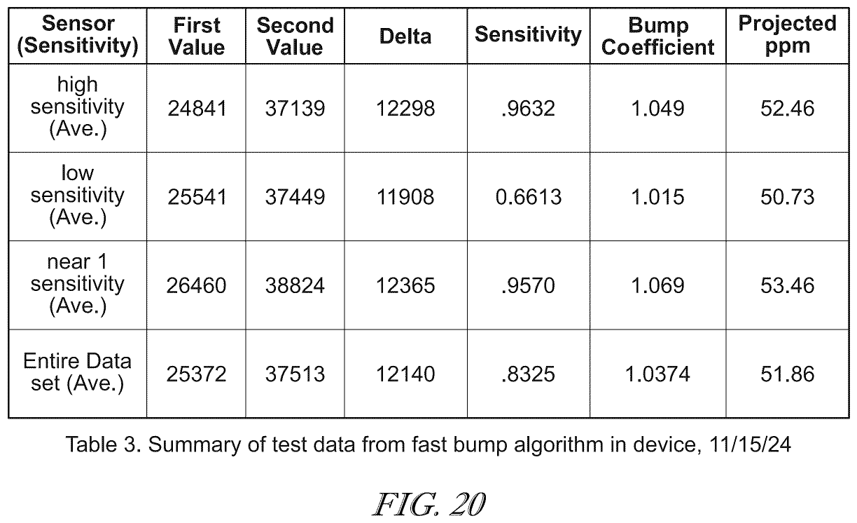

In some embodiments, bump testing of the system 100 is done with a calibration cup. For an illustrative example, the bump testing may be done using a 50 ppm calibration gas. FIGS. 18-20 show examples of the captured the ADC counts from testing using the rate zone measurements, as taken in steps 305, 306, and the projected actual ppm determined in step 312 using the early gas detection method 300. In the example for the results of shown in FIGS. 18-20, the system 100 used to conduct the test were in a 2000 count per ppm system with the rate zone initiation at 20,000 ADC counts. The determined, projected concentrations were calculated using the multiplier derived from the modifier coefficient and sensor sensitivity, as described above. In FIGS. 18-20, a wide array of example sensor responses are represented in the data along with the coefficients and projected concentration for the data for a variety of simulated bump tests.

A typical alarm threshold scenario for a CO detector is 35 ppm corresponding to low alarm, 70 ppm corresponding to a high alarm, and 200 ppm corresponding to a high-high alarm. In an illustrative example, a sensor with a T90 of 15 seconds and a rate zone value of 11816 would project to the low alarm using the describe method 300. If the rate zone value was larger than the 11816 counts it would indicate a gas concentration above 50 ppm.

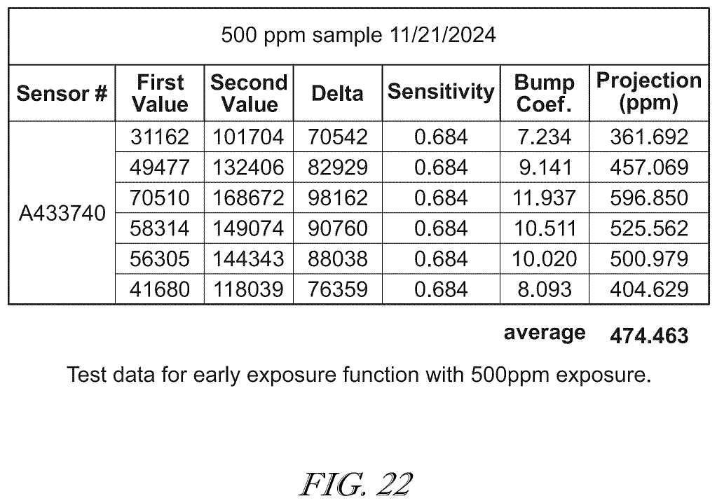

Testing for relatively higher concentrations of gas may be difficult because the high concentration gas always mixes with a certain amount of air in front of a face of the sensor 120. It is impossible to go from zero gas to a high concentration of gas at the face of the sensor 120 in a step function. With that said, shown in FIG. 22 is a sample of 500 ppm exposure with the early gas detection method 300 described within.

It is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed thereafter and equivalents thereof as well as additional items and equivalents thereof. Similarly, while a memory is recited as sometimes storing the aforementioned computer-executable instructions that are executed by the processor or controller, a person having skill in the art, after review of the entirety disclosed herein, will recognize that the computer-executable instructions may be hardcoded into the controller or processor, e.g., in the form of an application specific integrated circuit (ASIC), field programmable gate array (FPGA), etc.

Although this disclosure refers to specific embodiments, it will be understood by those skilled in the art that various changes in form and detail may be made without departing from the subject matter set forth in the accompanying claims. For example, while the disclosure has been illustrated and described in detail in the drawings and the foregoing description, such illustration and description are to be considered illustrative or exemplary and not restrictive. The disclosure is not limited to the disclosed embodiments. From reading the present disclosure, other modifications will be apparent to a person skilled in the art. Such modifications may involve other features, which are already known in the art and may be used instead of or in addition to features already described herein. Such modifications may perform the describe method with fewer, additional, or different steps. Modifications may include performing the describe steps in a different order. In the claims, the word “comprising” does not exclude other elements or steps, and the indefinite article “a” or “an” does not exclude a plurality.

1. A method of determining a concentration of a gas comprising:
detecting a presence of a detected gas with a gas detector;
triggering a timer when a detected concentration of the gas reaches a predetermined level;
measuring the concentration of the gas at a first time;
measuring the concentration of the gas at a second time at an end of the timer;
determine a rate of change of the concentration of the gas from the first time to the second time;
comparing the rate of change to a known standard; and
determining, from the comparison of the rate of change to the known standard, an estimated actual concentration of the detected gas.

2-7. (canceled)

8. The method of claim 1, wherein the step of comparing the rate of change to the known standard comprises determining a rate of change of the concentration of the detected gas between the first time and the second time.

9. The method of claim 8, wherein the step of comparing the rate of change to the known standard comprises comparing the rate of change to a standard curve derived from the known standard.

10. The method of claim 9, wherein the step of determining the estimated actual concentration of the detected gas comprises projecting a path of the rate of change up the standard curve of the know standard to obtain the estimated actual concentration.

11. The method of claim 1 further comprising using the estimated actual concentration to trigger an alarm when the estimated actual concentration is above a predetermined threshold level.

12. The method of claim 11, further comprising triggering the alarm before the detected gas is measured to be at an actual concentration of the detected gas by a sensor of the gas detector.

13. The method of claim 12, wherein the method is used to bump test the gas detector.

14-15. (canceled)

16. The method of claim 8, wherein the step of determining the estimated actual concentration of the detected gas comprises taking into account a modifier coefficient identified from the determined rate of change.

17. The method of claim 16, wherein the step of determining the estimated actual concentration of the detected gas comprises taking into account a time variance due to a sensitivity of a sensor of the gas detector.

18. A method of bump testing a gas detection system:
exposing a sensor of the gas detection system to a calibration gas
detecting a presence of the calibration gas with a sensor of the gas detection system;
triggering a timer when a detected concentration of the calibration gas reaches a predetermined level;
measuring the concentration of the calibration gas at a first time;
measuring the concentration of the calibration gas at a second time at an end of the timer;
determine a rate of change of the concentration of the calibration gas from the first time to the second time;
comparing the rate of change to a known standard;
determining, from the comparison of the rate of change to the known standard, an estimated actual concentration of the calibration gas; and
triggering a response of the gas detection system when the estimated actual concentration of the calibration gas exceeds a predetermined setpoint.

19-22. (canceled)

23. The method of claim 18, wherein the step of comparing the rate of change to the known standard comprises obtaining the known standard from a normalized curve for a known gas concentration.

24. The method of claim 23, wherein the step of obtaining the known standard from the normalized curve comprises deriving a standard curve by applying a tangent line approximation to the normalized curve.

25. The method of claim 18, wherein the step of comparing the rate of change to the known standard comprises determining a rate of change of the concentration of the calibration gas between the first time and the second time.

26-30. (canceled)

31. The method of claim 18, wherein the step of determining the estimated actual concentration of the detected gas comprises taking into account a time variance due to a sensitivity of a sensor of the gas detector.

32. A method of determining a concentration of a gas comprising:
detecting a presence of a detected gas with a gas detector;
triggering a timer when a detected concentration of the gas reaches a predetermined level;
measuring the concentration of the gas at a first time;
measuring the concentration of the gas at a second time at an end of the timer;
determine a rate of change of the concentration of the gas from the first time to the second time;
comparing the rate of change to a known standard;
determining, from the comparison of the rate of change to the known standard, an estimated actual concentration of the detected gas; and
trigger an early warning alarm when the estimated actual concentration is above a predetermined threshold level.

33. The method of claim 32, further comprising triggering the alarm before the detected gas is measured to be at an actual concentration of the detected gas by a sensor of the gas detector.

34-39. (canceled)

40. The method of claim 32, wherein the step of comparing the rate of change to the known standard comprises determining a rate of change of the concentration of the detected gas between the first time and the second time.

41. The method of claim 40, wherein the step of comparing the rate of change to the known standard comprises comparing the rate of change to a standard curve derived from the known standard.

42. The method of claim 41, wherein the step of determining the estimated actual concentration of the detected gas comprises projecting a path of the rate of change up the standard curve of the know standard to obtain the estimated actual concentration.

43-45. (canceled)

46. The method of claim 32, wherein the step of determining the estimated actual concentration of the detected gas comprises taking into account a time variance due to a sensitivity of a sensor of the gas detector.
